(19) United States
(12) Patent Application Publication
(10) Pub. No.: US 2006/0110330 A1
(43) Pub. Date: May 25, 2006

(54) Inhalable pharmaceutical compositions containing an anticholinergic, formoterol, and a steroid

(75) Inventor: Michael P. Pieper, Biberach (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 11/274,648

(22) Filed: Nov. 15, 2005

(30) Foreign Application Priority Data
Nov. 23, 2004 (DE) 10 2004 056 578

(51) Int. Cl. A61L 9/04; A61K 31/573; A61K 31/4745

(52) U.S. Cl. 424/45; 514/171; 514/291

(57) ABSTRACT

A pharmaceutical composition containing an anticholinergic of formula 1 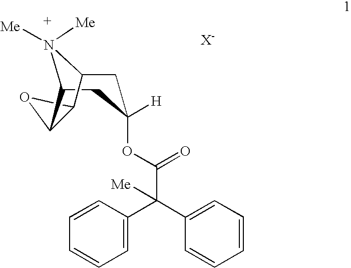
wherein X⁻ is an anion; a formoterol salt selected from formoterol fumarate and formoterol hemifumarate, or a hydrate, solvate, enantiomer, or mixtures of enantiomers thereof; and a steroid selected from ciclesonide, budesonide, and mometasone furoate, or a solvate or hydrate thereof.

### RELATED APPLICATIONS

This application claims priority to German Application No. DE 10 2004 056 578.3, filed Nov. 23, 2004, which is hereby incorporated by reference in its entirety.

### FIELD OF THE INVENTION

The present invention relates to novel pharmaceutical compositions based on a new anticholinergic, formoterol salts and a corticosteroid, processes for preparing them, and their use in the treatment of respiratory complaints.

### SUMMARY OF THE INVENTION

The present invention relates to novel pharmaceutical compositions containing an anticholinergic of formula 1 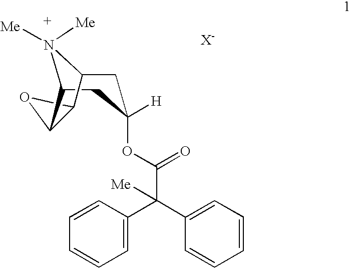
wherein X⁻ denotes an anion selected from among chloride, bromide, and methanesulfonate, preferably bromide;
a formoterol salt selected from among formoterol fumarate and formoterol hemifumarate, optionally in the form of the hydrates and/or solvates thereof, and optionally in the form of one of the respective enantiomers or mixtures of enantiomers thereof; and
a steroid selected from among ciclesonide, budesonide, and mometasone furoate, optionally each in the form of the hydrates and/or solvates thereof.

The salts of formula 1 are known from International Patent Application WO 02/32899, corresponding to U.S. Pat. No. 6,706,726, each of which is hereby incorporated by reference. Any reference to the salts of formula 1 includes a reference to any hydrates and solvates thereof which may be obtained.

Within the scope of the present patent application, an explicit reference to the pharmacologically active cation of formula 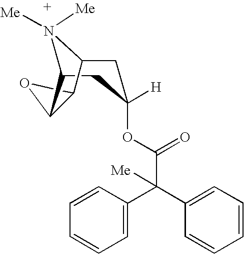
can be recognized by the use of the designation 1′. Any reference to compounds 1 naturally includes a reference to the cation 1′.

Any reference to the steroids ciclesonide, budesonide, and mometasone furoate within the scope of the present invention includes a reference to salts or derivatives which may be formed from the steroids. Examples of possible salts or derivatives include: sodium salts, sulfobenzoates, phosphates, isonicotinates, acetates, propionates, dihydrogen phosphates, palmitates, pivalates, or furoates. In some cases the steroids mentioned may also occur in the form of their hydrates, and in the case of mometasone furoate, particular importance attaches to mometasone furoate monohydrate.

The formoterol salt may optionally be present in enantiomerically pure form. Enantiomers which may be used according to the invention are selected from among salts of R,R-formoterol, S,S-formoterol, R,S-formoterol and S,R-formoterol, while the enantiomeric salts of R,R-formoterol are of particular importance. The formoterol salt may optionally be used in the form of the hydrates and/or solvates thereof. According to the invention formoterol fumarate dihydrate and formoterol hemifumarate monohydrate are of exceptional importance.

The pharmaceutical combinations according to the invention are administered by inhalation according to the invention. Suitable inhalable powders packed into suitable capsules (inhalettes) may preferably be administered using suitable powder inhalers according to the invention.

In one aspect, therefore, the present invention relates to a pharmaceutical composition which contains a combination of 1, formoterol salt, and one of the steroids ciclesonide, budesonide, or mometasone furoate.

In another aspect the present invention relates to a pharmaceutical kit which contains the abovementioned ingredients in separate pharmaceutical formulations.

In another aspect the present invention relates to a medicament which contains, in addition to therapeutically effective amounts of 1, formoterol salt, and ciclesonide, budesonide, or mometasone furoate, a pharmaceutically acceptable carrier. Particularly preferred medicaments contain, in addition to a pharmaceutically acceptable excipient or carrier, the following pharmaceutical combinations:
1′-bromide, formoterol hemifumarate monohydrate, and ciclesonide;
1′-bromide, formoterol hemifumarate monohydrate, and budesonide;
1′-bromide, formoterol hemifumarate monohydrate, and mometasone furoate monohydrate;
1′-bromide, formoterol fumarate dihydrate and ciclesonide;
1′-bromide, formoterol fumarate dihydrate and budesonide; or
1′-bromide, formoterol fumarate dihydrate and mometasone furoate monohydrate.

Of particular interest according to the invention are pharmaceutical combinations in which the formoterol salt is present in the form of formoterol hemifumarate, preferably in the form of formoterol hemifumarate monohydrate.

### BRIEF DESCRIPTION OF THE FIGURE

FIG. 1 shows a particularly preferred inhaler for using the pharmaceutical combination according to the invention in inhalettes.

### DETAILED DESCRIPTION OF THE INVENTION

The present invention further relates to the use of the abovementioned pharmaceutical combinations for preparing a pharmaceutical composition containing therapeutically effective quantities of the three ingredients for treating inflammatory and/or obstructive diseases of the respiratory tract, particularly asthma and/or chronic obstructive pulmonary disease (COPD), by simultaneous or successive administration. In addition the pharmaceutical combinations according to the invention may be used to prepare a drug for treating cystic fibrosis or allergic alveolitis (farmer's lung), for example, by simultaneous or successive administration. The combinations of active substances according to the invention will not be used only if treatment with one of the pharmaceutically active ingredients is contraindicated.

The present invention also relates to the simultaneous or successive use of therapeutically effective doses of the combination of the above pharmaceutical compositions for treating inflammatory or obstructive diseases of the respiratory tract, particularly asthma and/or chronic obstructive pulmonary disease (COPD), provided that treatment with steroids or betamimetics is not contraindicated from a therapeutic point of view, by simultaneous or successive administration. The invention further relates to the simultaneous or successive use of therapeutically effective doses of the combination of the above pharmaceutical compositions for treating cystic fibrosis or allergic alveolitis (farmer's lung), for example.

Preferred pharmaceutical combinations according to the invention which contain ciclesonide as steroid are preferably administered according to the invention such that 15 μg to 800 μg, preferably 50 μg to 400 μg, particularly 50 μg to 200 μg, of active substance 1, preferably in the form of the bromide, 3 μg to 20 μg, preferably 4 μg to 10 μg, of formoterol salt, and 50 μg to 400 μg, preferably 100 μg to 400 μg, of ciclesonide are given once or twice a day.

Preferred pharmaceutical combinations according to the invention which contain budesonide as steroid are preferably administered according to the invention such that 15 μg to 800 μg, preferably 50 μg to 400 μg, particularly 50 μg to 200 μg, of active substance 1, preferably in the form of the bromide, 3 μg to 20 μg, preferably 4 μg to 10 μg, of formoterol salt, and 200 μg to 800 μg, preferably 300 μg to 500 μg, of budesonide are given once or twice a day.

Preferred pharmaceutical combinations according to the invention which contain mometasone furoate as steroid are preferably administered according to the invention such that 15 μg to 800 μg, preferably 50 μg to 400 μg, particularly 50 μg to 200 μg, of active substance 1, preferably in the form of its bromide, 3 μg to 20 μg, preferably 4 μg to 10 μg, of formoterol salt, and 200 μg to 800 μg, preferably 300 μg to 500 μg, of mometasone furoate are given once or twice a day.

The active substance combinations according to the invention are preferably administered by inhalation. For this purpose, ingredients have to be made available in forms suitable for inhalation.

Inhalable preparations include, according to the invention, inhalable powders, propellant-containing metered-dose aerosols or propellant-free inhalable solutions. Inhalable powders according to the invention containing the abovementioned combinations of active substances may consist of the active substances on their own or of a mixture of the active substances with physiologically acceptable excipients. Within the scope of the present invention the term carrier may optionally be used instead of the term excipient.

Within the scope of the present invention, the term propellant-free inhalable solutions also includes concentrates or sterile ready-to-use inhalable solutions. The preparations according to the invention may contain the combination of active substances either together in one, in two or in three separate formulations. These formulations which may be used within the scope of the present invention are described in more detail in the next part of the specification.

A) Inhalable Powders

The inhalable powders according to the invention may contain the abovementioned active substances either on their own or in admixture with suitable physiologically acceptable excipients. If the active substances are present in admixture with physiologically acceptable excipients, the following physiologically acceptable excipients may be used to prepare these inhalable powders according to the invention: monosaccharides (e.g., glucose or arabinose), disaccharides (e.g., lactose, saccharose, maltose, trehalose), oligo- and polysaccharides (e.g., dextran), polyalcohols (e.g., sorbitol, mannitol, xylitol), salts (e.g., sodium chloride, calcium carbonate) or mixtures of these excipients. Preferably, mono- or disaccharides are used, while the use of lactose or glucose is preferred, particularly, but not exclusively, in the form of their hydrates. For the purposes of the invention, lactose is the particularly preferred excipient, while lactose monohydrate is most particularly preferred.

Within the scope of the inhalable powders according to the invention the excipients have a maximum average particle size of up to 250 μm, preferably between 10 and 150 μm, most preferably between 15 and 80 μm. It may sometimes seem appropriate to add finer excipient fractions with an average particle size of 1 to 9 μm to the excipient mentioned above. These finer excipients are also selected from the group of possible excipients listed hereinbefore. Finally, in order to prepare the inhalable powders according to the invention, the micronized active substances, preferably with an average particle size of 0.5 to 10 μm, more preferably from 1 to 5 μm, are added to the excipient mixture. Processes for producing the inhalable powders according to the invention by grinding and micronizing and by finally mixing the ingredients together are known from the prior art. The inhalable powders according to the invention may be prepared and administered either in the form of a single powder mixture which contains all three active substances or in the form of separate inhalable powders which contain only two or only one of the abovementioned active substances.

The inhalable powders according to the invention may be administered using inhalers known from the prior art.

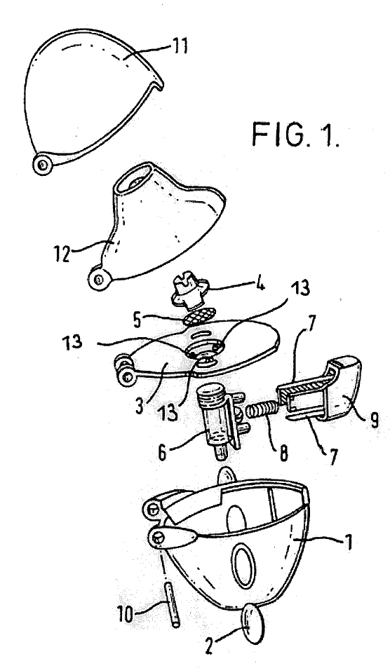

Inhalable powders according to the invention which contain one or more physiologically acceptable excipients in addition to the abovementioned active substances may be administered, for example, by means of inhalers which deliver a single dose from a supply using a measuring chamber as described in U.S. Pat. No. 4,570,630, or by other means as described in DE 36 25 685 A, each of which is incorporated by reference. Preferably, the inhalable powders according to the invention which contain physiologically acceptable excipients in addition to the abovementioned active substances are packed into capsules (to produce so-called inhalettes) which are used in inhalers as described, for example, in WO 94/28958. A particularly preferred inhaler for using the pharmaceutical combination according to the invention in inhalettes is shown in FIG. 1. This inhaler (HANDIHALER®) for inhaling powdered pharmaceutical compositions from capsules is characterized by a housing 1 containing two windows 2, a deck 3 in which there are air inlet ports and which is provided with a screen 5 secured over a screen housing 4, an inhalation chamber 6 connected to the deck 3 on which there is a push button 9 provided with two sharpened pins 7 and movable counter to a spring 8, and a mouthpiece 12 which is connected to the housing 1, the deck 3 and a cover 11 via a spindle 10 to enable it to be flipped open or shut, and air holes 13 for adjusting the flow resistance.

If the inhalable powders according to the invention are to be packed into capsules (inhalettes) for the preferred use described above, the quantities packed into each capsule should be 1 to 30 mg, preferably 3 to 20 mg, more particularly 5 to 10 mg of inhalable powder per capsule. These capsules contain, according to the invention, either together or separately, the doses of the abovementioned active substances mentioned hereinbefore for each single dose.

B) Propellant-Containing Inhalation Aerosols

Inhalation aerosols containing propellant gas according to the invention may contain the abovementioned active substances dissolved in the propellant gas or in dispersed form. The abovementioned active substances may be present in separate formulations or in a single preparation, in which the abovementioned active substances are either each dissolved, each dispersed or only one or two of the components are dissolved and the other is or are dispersed. The propellant gases which may be used to prepare the inhalation aerosols according to the invention are known from the prior art. Suitable propellant gases are selected from among hydrocarbons such as n-propane, n-butane, or isobutane and halohydrocarbons such as fluorinated derivatives of methane, ethane, propane, butane, cyclopropane, or cyclobutane. The propellant gases mentioned above may be used on their own or in mixtures thereof. Particularly preferred propellant gases are halogenated alkane derivatives selected from TG134a and TG227 and mixtures thereof.

The propellant-driven inhalation aerosols according to the invention may also contain other ingredients such as co-solvents, stabilizers, surfactants, antioxidants, lubricants, and pH adjusters. All these ingredients are known in the art.

The propellant-driven inhalation aerosols according to the invention may contain up to 5 wt.-% of the abovementioned active substances. Aerosols according to the invention contain, for example, 0.002 to 5 wt.-%, 0.01 to 3 wt.-%, 0.015 to 2 wt.-%, 0.1 to 2 wt.-%, 0.5 to 2 wt.-% or 0.5 to 1 wt.-% of one or more of the abovementioned active substances. If the active substances are present in dispersed form, the particles of active substance preferably have an average particle size of up to 10 μm, preferably from 0.1 to 5 μm, more preferably from 1 to 5 μm.

The propellant-driven inhalation aerosols according to the invention mentioned above may be administered using inhalers known in the art (MDIs or metered dose inhalers). Accordingly, in another aspect, the present invention relates to pharmaceutical compositions in the form of propellant-driven aerosols as hereinbefore described combined with one or more inhalers suitable for administering these aerosols. In addition, the present invention relates to inhalers which are characterized in that they contain the propellant gas-containing aerosols described above according to the invention. The present invention also relates to cartridges which are fitted with a suitable valve and can be used in a suitable inhaler and which contain one of the abovementioned propellant gas-containing inhalation aerosols according to the invention. Suitable cartridges and methods of filling these cartridges with the inhalable aerosols containing propellant gas according to the invention are known from the prior art.

C) Propellant-Free Inhalable Solutions or Suspensions

It is particularly preferred to use the active substance combination according to the invention in the form of propellant-free inhalable solutions and suspensions. The solvent used may be an aqueous or alcoholic, preferably an ethanolic solution. The solvent may be water on its own or a mixture of water and ethanol. The relative proportion of ethanol compared with water is not limited but the maximum is preferably up to 70 percent by volume, more particularly up to 60 percent by volume and most preferably up to 30 percent by volume. The remainder of the volume is made up of water. The solutions or suspensions containing the abovementioned active substances, separately or together, are adjusted to a pH of 2 to 7, preferably 2 to 5, using suitable acids. The pH may be adjusted using acids selected from inorganic or organic acids. Examples of particularly suitable inorganic acids include hydrochloric acid, hydrobromic acid, nitric acid, sulfuric acid, and/or phosphoric acid. Examples of particularly suitable organic acids include ascorbic acid, citric acid, malic acid, tartaric acid, maleic acid, succinic acid, fumaric acid, acetic acid, formic acid, and/or propionic acid etc. Preferred inorganic acids are hydrochloric and sulfuric acids. It is also possible to use the acids which have already formed an acid addition salt with one of the active substances. Of the organic acids, ascorbic acid, fumaric acid, and citric acid are preferred. If desired, mixtures of the above acids may be used, particularly in the case of acids which have other properties in addition to their acidifying qualities, e.g., as flavorings, antioxidants or complexing agents, such as citric acid or ascorbic acid, for example. According to the invention, it is particularly preferred to use hydrochloric acid to adjust the pH.

According to the invention, the addition of edetic acid (EDTA) or one of the known salts thereof, sodium edetate, as stabilizer or complexing agent is unnecessary in the present formulation. Other embodiments may contain this compound or these compounds. In a preferred embodiment the content based on sodium edetate is less than 100 mg/100 mL, preferably less than 50 mg/100 mL, more preferably less than 20 mg/100 mL. Generally, inhalable solutions in which the content of sodium edetate is from 0 to 10 mg/100 mL are preferred.

Co-solvents and/or other excipients may be added to the propellant-free inhalable solutions according to the invention. Preferred co-solvents are those which contain hydroxyl groups or other polar groups, e.g., alcohols—particularly isopropyl alcohol, glycols—particularly propylene glycol, polyethylene glycol, polypropylene glycol, glycol ether, glycerol, polyoxyethylene alcohols and polyoxyethylene fatty acid esters. The terms excipients and additives in this context denote any pharmacologically acceptable substance which is not an active substance but which can be formulated with the active substance or substances in the pharmacologically suitable solvent in order to improve the qualitative properties of the active substance formulation. Preferably, these substances have no pharmacological effect or, in connection with the desired therapy, no appreciable or at least no undesirable pharmacological effect. The excipients and additives include, for example, surfactants such as soya lecithin, oleic acid, sorbitan esters, such as polysorbates, polyvinylpyrrolidone, other stabilizers, complexing agents, antioxidants, and/or preservatives which guarantee or prolong the shelf life of the finished pharmaceutical formulation, flavorings, vitamins, and/or other additives known in the art. The additives also include physiologically acceptable salts such as sodium chloride as isotonic agents.

The preferred excipients include antioxidants such as ascorbic acid, for example, provided that it has not already been used to adjust the pH, vitamin A, vitamin E, tocopherols and similar vitamins and provitamins occurring in the human body.

Preservatives may be used to protect the formulation from contamination with pathogens. Suitable preservatives are those which are known in the art, particularly cetyl pyridinium chloride, benzalkonium chloride, or benzoic acid or benzoates such as sodium benzoate in the concentration known from the prior art. The preservatives mentioned above are preferably present in concentrations of up to 50 mg/100 mL, more preferably between 5 and 20 mg/100 mL.

Preferred formulations contain, in addition to the solvent water and the combination of active substances, only benzalkonium chloride and sodium edetate. In another preferred embodiment, no sodium edetate is present.

The propellant-free inhalable solutions according to the invention are administered in particular using inhalers of the kind which are capable of nebulizing a small amount of a liquid formulation in the required therapeutic dose within a few seconds to produce an aerosol suitable for therapeutic inhalation. Within the scope of the present invention, preferred nebulizers are those in which a quantity of less than 100 μL, preferably less than 50 μL, more preferably between 20 and 30 μL of active substance solution can be nebulized in preferably one spray action to form an aerosol with an average particle size of less than 20 μm, preferably less than 10 μm, in such a way that the inhalable part of the aerosol corresponds to the therapeutically effective quantity.

An apparatus of this kind for propellant-free delivery of a metered quantity of a liquid pharmaceutical composition for inhalation is described for example in International Patent Application WO 91/14468 and also in WO 97/12687 (cf. in particular FIGS. 6a and 6b). The nebulizers (devices) described therein are also known by the name RESPIMAT®.

This nebulizer (RESPIMAT®) can advantageously be used to produce the inhalable aerosols according to the invention containing the combination of active substances according to the invention. Because of its cylindrical shape and handy size of less than 9 to 15 cm long and 2 to 4 cm wide, this device can be carried at all times by the patient. The nebulizer sprays a defined volume of the pharmaceutical formulation at high pressures through small nozzles so as to produce inhalable aerosols.

The preferred atomizer essentially consists of an upper housing part, a pump housing, a nozzle, a locking mechanism, a spring housing, a spring and a storage container, characterized by
a pump housing which is secured in the upper housing part and which comprises at one end a nozzle body with the nozzle or nozzle arrangement,
a hollow plunger with valve body,
a power takeoff flange in which the hollow plunger is secured and which is located in the upper housing part,
a locking mechanism situated in the upper housing part,
a spring housing with the spring contained therein, which is rotatably mounted on the upper housing part by means of a rotary bearing, and
a lower housing part which is fitted onto the spring housing in the axial direction.

The hollow plunger with valve body corresponds to a device disclosed in WO 97/12687. It projects partially into the cylinder of the pump housing and is axially movable within the cylinder. Reference is made in particular to FIGS. 1 to 4, especially FIG. 3, and the relevant parts of the description. The hollow plunger with valve body exerts a pressure of 5 to 60 MPa (about 50 to 600 bar), preferably 10 to 60 MPa (about 100 to 600 bar) on the fluid, the measured amount of active substance solution, at its high pressure end at the moment when the spring is actuated. Volumes of 10 to 50 microliters are preferred, while volumes of 10 to 20 microliters are particularly preferred and a volume of 15 microliters per spray is most particularly preferred.

The valve body is preferably mounted at the end of the hollow plunger facing the valve body.

The nozzle in the nozzle body is preferably microstructured, i.e., produced by microtechnology. Microstructured valve bodies are disclosed, for example, in WO 94/07607; reference is hereby made to the contents of this specification, particularly FIG. 1 therein and the associated description.

The nozzle body consists for example of two sheets of glass and/or silicon firmly joined together, at least one of which has one or more microstructured channels which connect the nozzle inlet end to the nozzle outlet end. At the nozzle outlet end there is at least one round or non-round opening 2 to 10 microns deep and 5 to 15 microns wide, the depth preferably being 4.5 to 6.5 microns while the length is preferably 7 to 9 microns.

In the case of a plurality of nozzle openings, preferably two, the directions of spraying of the nozzles in the nozzle body may extend parallel to one another or may be inclined relative to one another in the direction of the nozzle opening. In a nozzle body with at least two nozzle openings at the outlet end the directions of spraying may be at an angle of 20 to 160° to one another, preferably 60 to 150°, most preferably 80 to 100°. The nozzle openings are preferably arranged at a spacing of 10 to 200 microns, more preferably at a spacing of 10 to 100 microns, most preferably 30 to 70 microns. Spacings of 50 microns are most preferred. The directions of spraying will therefore meet in the vicinity of the nozzle openings.

The liquid pharmaceutical preparation strikes the nozzle body with an entry pressure of up to 600 bar, preferably 200 to 300 bar, and is atomized into an inhalable aerosol through the nozzle openings. The preferred particle or droplet sizes of the aerosol are up to 20 microns, preferably 3 to 10 microns.

The locking mechanism contains a spring, preferably a cylindrical helical compression spring, as a store for the mechanical energy. The spring acts on the power takeoff flange as an actuating member the movement of which is determined by the position of a locking member. The travel of the power takeoff flange is precisely limited by an upper and lower stop. The spring is preferably biased, via a power step-up gear, e.g., a helical thrust gear, by an external torque which is produced when the upper housing part is rotated counter to the spring housing in the lower housing part. In this case, the upper housing part and the power takeoff flange have a single or multiple V-shaped gear.

The locking member with engaging locking surfaces is arranged in a ring around the power takeoff flange. It consists, for example, of a ring of plastic or metal which is inherently radially elastically deformable. The ring is arranged in a plane at right angles to the atomizer axis. After the biasing of the spring, the locking surfaces of the locking member move into the path of the power takeoff flange and prevent the spring from relaxing. The locking member is actuated by means of a button. The actuating button is connected or coupled to the locking member. In order to actuate the locking mechanism, the actuating button is moved parallel to the annular plane, preferably into the atomizer; this causes the deformable ring to deform in the annular plane. Details of the construction of the locking mechanism are given in WO 97/20590.

The lower housing part is pushed axially over the spring housing and covers the mounting, the drive of the spindle and the storage container for the fluid.

When the atomizer is actuated the upper housing part is rotated relative to the lower housing part, the lower housing part taking the spring housing with it. The spring is thereby compressed and biased by means of the helical thrust gear and the locking mechanism engages automatically. The angle of rotation is preferably a whole-number fraction of 360°, e.g., 180°. At the same time as the spring is biased, the power takeoff part in the upper housing part is moved along by a given distance, the hollow plunger is withdrawn inside the cylinder in the pump housing, as a result of which some of the fluid is sucked out of the storage container and into the high pressure chamber in front of the nozzle.

If desired, a number of exchangeable storage containers which contain the fluid to be atomized may be pushed into the atomizer one after another and used in succession. The storage container contains the aqueous aerosol preparation according to the invention.

The atomizing process is initiated by pressing gently on the actuating button. As a result, the locking mechanism opens up the path for the power takeoff member. The biased spring pushes the plunger into the cylinder of the pump housing. The fluid leaves the nozzle of the atomizer in atomized form.

Further details of construction are disclosed in PCT Applications WO 97/12683 and WO 97/20590, to which reference is hereby made.

The components of the atomizer (nebulizer) are made of a material which is suitable for its purpose. The housing of the atomizer and, if its operation permits, other parts as well, are preferably made of plastics, e.g., by injection molding. For medicinal purposes, physiologically safe materials are used.

FIGS. 6a/b of WO 97/12687, to which reference is explicitly made at this point, show the nebulizer (RESPIMAT®) which can advantageously be used for inhaling the aqueous aerosol preparations according to the invention.

FIG. 6a of WO 97/12687 shows a longitudinal section through the atomizer with the spring biased while FIG. 6b of WO 97/12687 shows a longitudinal section through the atomizer with the spring relaxed.

The upper housing part (51) contains the pump housing (52) on the end of which is mounted the holder (53) for the atomizer nozzle. In the holder is the nozzle body (54) and a filter (55). The hollow plunger (57) fixed in the power takeoff flange (56) of the locking mechanism projects partially into the cylinder of the pump housing. At its end the hollow plunger carries the valve body (58). The hollow plunger is sealed off by means of the seal (59). Inside the upper housing part is the stop (60) on which the power takeoff flange abuts when the spring is relaxed. On the power takeoff flange is the stop (61) on which the power takeoff flange abuts when the spring is biased. After the biasing of the spring the locking member (62) moves between the stop (61) and a support (63) in the upper housing part. The actuating button (64) is connected to the locking member. The upper housing part ends in the mouthpiece (65) and is sealed off by means of the protective cover (66) which can be placed thereon.

The spring housing (67) with compression spring (68) is rotatably mounted on the upper housing part by means of the snap-in lugs (69) and rotary bearing. The lower housing part (70) is pushed over the spring housing. Inside the spring housing is the exchangeable storage container (71) for the fluid (72) which is to be atomized. The storage container is sealed off by the stopper (73) through which the hollow plunger projects into the storage container and is immersed at its end in the fluid (supply of active substance solution). The spindle (74) for the mechanical counter is mounted in the covering of the spring housing. At the end of the spindle facing the upper housing part is the drive pinion (75). The slider (76) sits on the spindle.

The nebulizer described above is suitable for nebulizing the aerosol preparations according to the invention to produce an aerosol suitable for inhalation.

If the formulation according to the invention is nebulized using the technology described above (Respimat®) the quantity delivered should correspond to a defined quantity with a tolerance of not more than 25%, preferably 20% of this amount in at least 97%, preferably at least 98% of all operations of the inhaler (spray actuations). Preferably, between 5 and 30 mg of formulation, most preferably between 5 and 20 mg of formulation are delivered as a defined mass on each actuation.

However, the formulation according to the invention may also be nebulized by means of inhalers other than those described above, e.g., jet stream inhalers.

Accordingly, in a further aspect, the invention relates to pharmaceutical formulations in the form of propellant-free inhalable solutions or suspensions as described above combined with a device suitable for administering these formulations, preferably in conjunction with the RESPIMAT®. Preferably, the invention relates to propellant-free inhalable solutions or suspensions characterized by the combination of active substances according to the invention in conjunction with the device known by the name RESPIMAT®. In addition, the present invention relates to the abovementioned devices for inhalation, preferably the RESPIMAT®, characterized in that they contain the propellant-free inhalable solutions or suspensions according to the invention as described hereinbefore.

The propellant-free inhalable solutions or suspensions according to the invention may take the form of concentrates or sterile ready-to-use inhalable solutions or suspensions, as well as the abovementioned solutions and suspensions designed for use in a RESPIMAT®. Ready-to-use formulations may be produced from the concentrates, for example, by the addition of isotonic saline solutions. Sterile ready-to-use formulations may be administered using energy-operated fixed or portable nebulizers which produce inhalable aerosols by means of ultrasound or compressed air by the Venturi principle or other principles.

Accordingly, in another aspect, the present invention relates to pharmaceutical compositions in the form of propellant-free inhalable solutions or suspensions as described hereinbefore which take the form of concentrates or sterile ready-to-use formulations, combined with a device suitable for administering these solutions, characterized in that the device is an energy-operated free-standing or portable nebulizer which produces inhalable aerosols by means of ultrasound or compressed air by the Venturi principle or other methods.

The Examples which follow serve to illustrate the present invention in more detail without restricting the scope of the invention to the following embodiments by way of example.

### EXAMPLES OF FORMULATIONS

A) Inhalable Powders

The Table that follows describes examples of inhalable powders according to the invention which contain 1′-bromide, formoterol hemifumarate monohydrate, budesonide, and lactose monohydrate as excipient.

| | 1′-salt | steroid | formoterol salt | excipient |
|---|---|---|---|---|
| Example | [μg] | [μg] | [μg] | [μg] |
| 1 | 50 | 400 | 5 | 4545 |
| 2 | 75 | 400 | 5 | 4520 |
| 3 | 100 | 400 | 5 | 4495 |
| 4 | 200 | 400 | 5 | 4395 |
| 5 | 400 | 400 | 5 | 4195 |
| 6 | 800 | 400 | 5 | 3795 |
| 7 | 50 | 300 | 5 | 4645 |
| 8 | 75 | 300 | 5 | 4620 |
| 9 | 100 | 300 | 5 | 4595 |
| 10 | 200 | 300 | 5 | 4495 |
| 11 | 400 | 300 | 5 | 4295 |
| 12 | 800 | 300 | 5 | 3895 |
| 13 | 50 | 500 | 5 | 4445 |
| 14 | 75 | 500 | 5 | 4420 |
| 15 | 100 | 500 | 5 | 4395 |
| 16 | 200 | 500 | 5 | 4295 |
| 17 | 400 | 500 | 5 | 4095 |
| 18 | 800 | 500 | 5 | 3695 |
| 19 | 50 | 400 | 10 | 4540 |
| 20 | 75 | 400 | 10 | 4515 |
| 21 | 100 | 400 | 10 | 4515 |
| 22 | 200 | 400 | 10 | 4390 |
| 23 | 400 | 400 | 10 | 4190 |
| 24 | 800 | 400 | 10 | 3790 |
| 25 | 50 | 300 | 10 | 4640 |
| 26 | 75 | 300 | 10 | 4615 |
| 27 | 100 | 300 | 10 | 4590 |
| 28 | 200 | 300 | 10 | 4490 |
| 29 | 400 | 300 | 10 | 4290 |
| 30 | 800 | 300 | 10 | 3890 |
| 31 | 50 | 500 | 10 | 4440 |
| 32 | 75 | 500 | 10 | 4415 |
| 33 | 100 | 500 | 10 | 4390 |
| 34 | 200 | 500 | 10 | 4290 |
| 35 | 400 | 500 | 10 | 4090 |
| 36 | 800 | 500 | 10 | 3690 |
| 37 | 50 | 400 | 15 | 4535 |
| 38 | 75 | 400 | 15 | 4510 |
| 39 | 100 | 400 | 15 | 4485 |
| 40 | 200 | 400 | 15 | 4385 |
| 41 | 400 | 400 | 15 | 4185 |
| 42 | 800 | 400 | 15 | 3785 |
| 43 | 50 | 300 | 15 | 4635 |
| 44 | 75 | 300 | 15 | 4610 |
| 45 | 100 | 300 | 15 | 4585 |
| 46 | 200 | 300 | 15 | 4485 |
| 47 | 400 | 300 | 15 | 4285 |
| 48 | 800 | 300 | 15 | 3885 |
| 49 | 50 | 500 | 15 | 4435 |
| 50 | 75 | 500 | 15 | 4410 |
| 51 | 100 | 500 | 15 | 4385 |
| 52 | 200 | 500 | 15 | 4285 |
| 53 | 400 | 500 | 15 | 4085 |
| 54 | 800 | 500 | 15 | 3685 |
| 55 | 50 | 400 | 7.5 | 4542.5 |
| 56 | 75 | 400 | 7.5 | 4517.5 |
| 57 | 100 | 400 | 7.5 | 4492.5 |
| 58 | 200 | 400 | 7.5 | 4392.5 |
| 59 | 400 | 400 | 7.5 | 4192.5 |
| 60 | 800 | 400 | 7.5 | 3792.5 |
| 61 | 50 | 300 | 7.5 | 4642.5 |
| 62 | 75 | 300 | 7.5 | 4617.5 |
| 63 | 100 | 300 | 7.5 | 4592.5 |
| 64 | 200 | 300 | 7.5 | 4492.5 |
| 65 | 400 | 300 | 7.5 | 4292.5 |
| 66 | 800 | 300 | 7.5 | 3892.5 |
| 67 | 50 | 500 | 7.5 | 4442.5 |
| 68 | 75 | 500 | 7.5 | 4417.5 |
| 69 | 100 | 500 | 7.5 | 4392.5 |
| 70 | 200 | 500 | 7.5 | 4292.5 |
| 71 | 400 | 500 | 7.5 | 4092.5 |
| 72 | 800 | 500 | 7.5 | 3692.5 |

The following Table describes examples of inhalable powders according to the invention which contain 1′-bromide, formoterol hemifumarate monohydrate, ciclesonide, and lactose monohydrate as excipient.

| | 1′-salt | steroid | formoterol salt | excipient |
|---|---|---|---|---|
| Example | [μg] | [μg] | [μg] | [μg] |
| 1 | 50 | 400 | 5 | 4545 |
| 2 | 75 | 400 | 5 | 4520 |
| 3 | 100 | 400 | 5 | 4495 |
| 4 | 200 | 400 | 5 | 4395 |
| 5 | 400 | 400 | 5 | 4195 |
| 6 | 800 | 400 | 5 | 3795 |
| 7 | 50 | 300 | 5 | 4645 |
| 8 | 75 | 300 | 5 | 4620 |
| 9 | 100 | 300 | 5 | 4595 |
| 10 | 200 | 300 | 5 | 4495 |
| 11 | 400 | 300 | 5 | 4295 |
| 12 | 800 | 300 | 5 | 3895 |
| 13 | 50 | 200 | 5 | 4745 |
| 14 | 75 | 200 | 5 | 4720 |
| 15 | 100 | 200 | 5 | 4695 |
| 16 | 200 | 200 | 5 | 4595 |
| 17 | 400 | 200 | 5 | 4395 |
| 18 | 800 | 200 | 5 | 3995 |
| 19 | 50 | 400 | 10 | 4540 |
| 20 | 75 | 400 | 10 | 4515 |
| 21 | 100 | 400 | 10 | 4515 |
| 22 | 200 | 400 | 10 | 4390 |
| 23 | 400 | 400 | 10 | 4190 |
| 24 | 800 | 400 | 10 | 3790 |
| 25 | 50 | 300 | 10 | 4640 |
| 26 | 75 | 300 | 10 | 4615 |
| 27 | 100 | 300 | 10 | 4590 |
| 28 | 200 | 300 | 10 | 4490 |
| 29 | 400 | 300 | 10 | 4290 |
| 30 | 800 | 300 | 10 | 3890 |
| 31 | 50 | 200 | 10 | 4740 |
| 32 | 75 | 200 | 10 | 4715 |
| 33 | 100 | 200 | 10 | 4690 |
| 34 | 200 | 200 | 10 | 4590 |
| 35 | 400 | 200 | 10 | 4390 |
| 36 | 800 | 200 | 10 | 3990 |
| 37 | 50 | 400 | 15 | 4535 |
| 38 | 75 | 400 | 15 | 4510 |
| 39 | 100 | 400 | 15 | 4485 |
| 40 | 200 | 400 | 15 | 4385 |
| 41 | 400 | 400 | 15 | 4185 |
| 42 | 800 | 400 | 15 | 3785 |
| 43 | 50 | 300 | 15 | 4635 |
| 44 | 75 | 300 | 15 | 4610 |
| 45 | 100 | 300 | 15 | 4585 |
| 46 | 200 | 300 | 15 | 4485 |
| 47 | 400 | 300 | 15 | 4285 |
| 48 | 800 | 300 | 15 | 3885 |
| 49 | 50 | 200 | 15 | 4735 |
| 50 | 75 | 200 | 15 | 4710 |
| 51 | 100 | 200 | 15 | 4685 |
| 52 | 200 | 200 | 15 | 4685 |
| 53 | 400 | 200 | 15 | 4485 |
| 54 | 800 | 200 | 15 | 3885 |
| 55 | 50 | 400 | 7.5 | 4542.5 |
| 56 | 75 | 400 | 7.5 | 4517.5 |
| 57 | 100 | 400 | 7.5 | 4492.5 |
| 58 | 200 | 400 | 7.5 | 4392.5 |
| 59 | 400 | 400 | 7.5 | 4192.5 |
| 60 | 800 | 400 | 7.5 | 3792.5 |
| 61 | 50 | 300 | 7.5 | 4642.5 |
| 62 | 75 | 300 | 7.5 | 4617.5 |
| 63 | 100 | 300 | 7.5 | 4592.5 |
| 64 | 200 | 300 | 7.5 | 4492.5 |
| 65 | 400 | 300 | 7.5 | 4292.5 |
| 66 | 800 | 300 | 7.5 | 3892.5 |
| 67 | 50 | 200 | 7.5 | 4742.5 |
| 68 | 75 | 200 | 7.5 | 4717.5 |
| 69 | 100 | 200 | 7.5 | 4692.5 |
| 70 | 200 | 200 | 7.5 | 4592.5 |
| 71 | 400 | 200 | 7.5 | 4392.5 |
| 72 | 800 | 200 | 7.5 | 3992.5 |

The following Table describes examples of inhalable powders according to the invention which contain 1′-bromide, formoterol hemifumarate monohydrate, mometasone furoate, and lactose monohydrate as excipient.

| | 1′-salt | steroid | formoterol salt | excipient |
|---|---|---|---|---|
| Example | [μg] | [μg] | [μg] | [μg] |
| 1 | 50 | 400 | 5 | 4545 |
| 2 | 75 | 400 | 5 | 4520 |
| 3 | 100 | 400 | 5 | 4495 |
| 4 | 200 | 400 | 5 | 4395 |
| 5 | 400 | 400 | 5 | 4195 |
| 6 | 800 | 400 | 5 | 3795 |
| 7 | 50 | 300 | 5 | 4645 |
| 8 | 75 | 300 | 5 | 4620 |
| 9 | 100 | 300 | 5 | 4595 |
| 10 | 200 | 300 | 5 | 4495 |
| 11 | 400 | 300 | 5 | 4295 |
| 12 | 800 | 300 | 5 | 3895 |
| 13 | 50 | 500 | 5 | 4445 |
| 14 | 75 | 500 | 5 | 4420 |
| 15 | 100 | 500 | 5 | 4395 |
| 16 | 200 | 500 | 5 | 4295 |
| 17 | 400 | 500 | 5 | 4095 |
| 18 | 800 | 500 | 5 | 3695 |
| 19 | 50 | 400 | 10 | 4540 |
| 20 | 75 | 400 | 10 | 4515 |
| 21 | 100 | 400 | 10 | 4515 |
| 22 | 200 | 400 | 10 | 4390 |
| 23 | 400 | 400 | 10 | 4190 |
| 24 | 800 | 400 | 10 | 3790 |
| 25 | 50 | 300 | 10 | 4640 |
| 26 | 75 | 300 | 10 | 4615 |
| 27 | 100 | 300 | 10 | 4590 |
| 28 | 200 | 300 | 10 | 4490 |
| 29 | 400 | 300 | 10 | 4290 |
| 30 | 800 | 300 | 10 | 3890 |
| 31 | 50 | 500 | 10 | 4440 |
| 32 | 75 | 500 | 10 | 4415 |
| 33 | 100 | 500 | 10 | 4390 |
| 34 | 200 | 500 | 10 | 4290 |
| 35 | 400 | 500 | 10 | 4090 |
| 36 | 800 | 500 | 10 | 3690 |
| 37 | 50 | 400 | 15 | 4535 |
| 38 | 75 | 400 | 15 | 4510 |
| 39 | 100 | 400 | 15 | 4485 |
| 40 | 200 | 400 | 15 | 4385 |
| 41 | 400 | 400 | 15 | 4185 |
| 42 | 800 | 400 | 15 | 3785 |
| 43 | 50 | 300 | 15 | 4635 |
| 44 | 75 | 300 | 15 | 4610 |
| 45 | 100 | 300 | 15 | 4585 |
| 46 | 200 | 300 | 15 | 4485 |
| 47 | 400 | 300 | 15 | 4285 |
| 48 | 800 | 300 | 15 | 3885 |
| 49 | 50 | 500 | 15 | 4435 |
| 50 | 75 | 500 | 15 | 4410 |
| 51 | 100 | 500 | 15 | 4385 |
| 52 | 200 | 500 | 15 | 4285 |
| 53 | 400 | 500 | 15 | 4085 |
| 54 | 800 | 500 | 15 | 3685 |
| 55 | 50 | 400 | 7.5 | 4542.5 |
| 56 | 75 | 400 | 7.5 | 4517.5 |
| 57 | 100 | 400 | 7.5 | 4492.5 |
| 58 | 200 | 400 | 7.5 | 4392.5 |
| 59 | 400 | 400 | 7.5 | 4192.5 |
| 60 | 800 | 400 | 7.5 | 3792.5 |
| 61 | 50 | 300 | 7.5 | 4642.5 |
| 62 | 75 | 300 | 7.5 | 4617.5 |
| 63 | 100 | 300 | 7.5 | 4592.5 |
| 64 | 200 | 300 | 7.5 | 4492.5 |
| 65 | 400 | 300 | 7.5 | 4292.5 |
| 66 | 800 | 300 | 7.5 | 3892.5 |
| 67 | 50 | 500 | 7.5 | 4442.5 |
| 68 | 75 | 500 | 7.5 | 4417.5 |
| 69 | 100 | 500 | 7.5 | 4392.5 |
| 70 | 200 | 500 | 7.5 | 4292.5 |
| 71 | 400 | 500 | 7.5 | 4092.5 |
| 72 | 800 | 500 | 7.5 | 3692.5 |

B) Propellant-Containing Inhalable Aerosols

The following Table describes examples of propellant-containing inhalable aerosols according to the invention which contain 1′-bromide, formoterol hemifumarate monohydrate, and budesonide. The amounts specified are percent by weight (based on the total formulation).

| | | | formo- | | soya | | propellant |
|---|---|---|---|---|---|---|---|
| Exam- | | ste- | terol | isopropyl | leci- | ethanol | gas |
| ple | 1′-salt | roid | salt | myristate | thin | (abs) | [ad 100] |
| 1 | 0.035 | 0.4 | 0.066 | — | 0.2 | — | TG134a/ |
| | | | | | | | TG227 |
| | | | | | | | (2:3) |
| 2 | 0.030 | 0.4 | 0.066 | — | 0.2 | — | TG227 |
| 3 | 0.025 | 0.4 | 0.033 | 0.1 | — | — | TG227 |
| 4 | 0.035 | 0.2 | 0.066 | — | 0.2 | — | TG134a/ |
| | | | | | | | TG227 |
| | | | | | | | (2:3) |
| 5 | 0.030 | 0.2 | 0.066 | 0.1 | — | — | TG227 |
| 6 | 0.025 | 0.2 | 0.033 | 0.1 | — | — | TG227 |
| 7 | 0.039 | 0.4 | 0.066 | 0.1 | — | 0.5 | TG134a/ |
| | | | | | | | TG227 |
| | | | | | | | (2:3) |
| 8 | 0.039 | 0.45 | 0.08 | — | 0.2 | 0.5 | TG227 |
| 9 | 0.039 | 0.4 | 0.033 | 0.1 | — | 0.5 | TG227 |
| 10 | 0.035 | 0.2 | 0.066 | 0.1 | — | 0.5 | TG134a/ |
| | | | | | | | TG227 |
| | | | | | | | (2:3) |

The following Table describes examples of propellant-containing inhalable aerosols according to the invention which contain 1′-bromide, formoterol hemifumarate monohydrate, and ciclesonide. The amounts specified are percent by weight (based on the total formulation).

| | | | formo- | | soya | | propellant |
|---|---|---|---|---|---|---|---|
| Exam- | | ste- | terol | isopropyl | leci- | ethanol | gas |
| ple | 1′-salt | roid | salt | myristate | thin | (abs) | [ad 100] |
| 1 | 0.035 | 0.4 | 0.066 | — | 0.2 | — | TG134a/ |
| | | | | | | | TG227 |
| | | | | | | | (2:3) |
| 2 | 0.030 | 0.4 | 0.066 | — | 0.2 | — | TG227 |
| 3 | 0.025 | 0.4 | 0.033 | 0.1 | — | — | TG227 |
| 4 | 0.025 | 0.2 | 0.066 | — | 0.2 | — | TG134a/ |
| | | | | | | | TG227 |
| | | | | | | | (2:3) |
| 5 | 0.030 | 0.2 | 0.066 | 0.1 | — | — | TG227 |
| 6 | 0.025 | 0.2 | 0.033 | 0.1 | — | — | TG227 |
| 7 | 0.020 | 0.4 | 0.066 | 0.1 | — | 0.5 | TG134a/ |
| | | | | | | | TG227 |
| | | | | | | | (2:3) |
| 8 | 0.035 | 0.45 | 0.08 | — | 0.2 | 0.5 | TG227 |
| 9 | 0.035 | 0.4 | 0.033 | 0.1 | — | 0.5 | TG227 |
| 10 | 0.025 | 0.2 | 0.066 | 0.1 | — | 0.5 | TG134a/ |
| | | | | | | | TG227 |
| | | | | | | | (2:3) |

The following Table describes examples of propellant-containing inhalable aerosols according to the invention which contain 1′-bromide, formoterol hemifumarate monohydrate, and mometasone furoate. The amounts specified are percent by weight (based on the total formulation).

| | | | formo- | | soya | | propellant |
|---|---|---|---|---|---|---|---|
| Exam- | | ste- | terol | isopropyl | leci- | ethanol | gas |
| ple | 1′-salt | roid | salt | myristate | thin | (abs) | [ad 100] |
| 1 | 0.035 | 0.4 | 0.066 | — | 0.2 | — | TG134a/ |
| | | | | | | | TG227 |
| | | | | | | | (2:3) |
| 2 | 0.030 | 0.4 | 0.066 | — | 0.2 | — | TG227 |
| 3 | 0.025 | 0.4 | 0.033 | 0.1 | — | — | TG227 |
| 4 | 0.035 | 0.2 | 0.066 | — | 0.2 | — | TG134a/ |
| | | | | | | | TG227 |
| | | | | | | | (2:3) |
| 5 | 0.030 | 0.2 | 0.066 | 0.1 | — | — | TG227 |
| 6 | 0.025 | 0.2 | 0.033 | 0.1 | — | — | TG227 |
| 7 | 0.039 | 0.4 | 0.066 | 0.1 | — | 0.5 | TG134a/ |
| | | | | | | | TG227 |
| | | | | | | | (2:3) |
| 8 | 0.039 | 0.45 | 0.08 | — | 0.2 | 0.5 | TG227 |
| 9 | 0.039 | 0.4 | 0.033 | 0.1 | — | 0.5 | TG227 |
| 10 | 0.035 | 0.2 | 0.066 | 0.1 | — | 0.5 | TG134a/ |
| | | | | | | | TG227 |
| | | | | | | | (2:3) |

We claim:

1. A pharmaceutical composition comprising an anticholinergic of formula 1 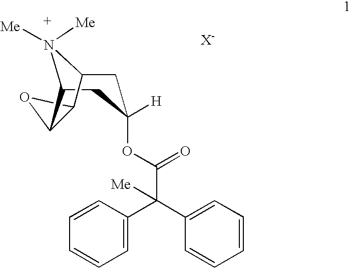
wherein X⁻ is an anion;
a formoterol salt selected from formoterol fumarate and formoterol hemifumarate, or a hydrate, solvate, enantiomer, or mixtures of enantiomers thereof; and
a steroid selected from ciclesonide, budesonide, and mometasone furoate, or a solvate or hydrate thereof.

2. The pharmaceutical composition according to claim 1, further comprising a physiologically acceptable excipient.

3. The pharmaceutical composition according to claim 1, wherein X⁻ is chloride, bromide, or methanesulfonate.

4. The pharmaceutical composition according to claim 1, wherein X⁻ is bromide.

5. The pharmaceutical composition according to claim 1, wherein the pharmaceutical composition is suitable for inhalation.

6. The pharmaceutical composition according to claim 4, wherein the pharmaceutical composition is an inhalable powder, a propellant-containing metered-dose aerosol, or a propellant-free inhalable solution or suspension.

7. The pharmaceutical composition according to claim 6, wherein the pharmaceutical composition is an inhalable powder comprising the anticholinergic, the formoterol salt, and the steroid in admixture with a physiologically acceptable excipient selected from monosaccharides, disaccharides, oligo- and polysaccharides, polyalcohols, salts, or mixtures thereof.

8. The pharmaceutical composition according to claim 6, wherein the pharmaceutical composition is a propellant-containing metered-dose aerosol comprising the anticholinergic, the formoterol salt, and the steroid in dissolved or dispersed form.

9. The pharmaceutical composition according to claim 8, wherein the propellant gas is a hydrocarbon or halohydrocarbon.

10. The pharmaceutical composition according to claim 9, wherein the propellant gas is selected from n-propane; n-butane; isobutane; or the chlorinated and/or fluorinated derivatives of methane, ethane, propane, butane, cyclopropane, or cyclobutane.

11. The pharmaceutical composition according to claim 8, wherein the propellant gas is selected from TG11, TG12, TG134a, TG227, or a mixture thereof.

12. The pharmaceutical composition according to claim 6, wherein the pharmaceutical composition is a propellant-free inhalable solution or suspension further comprising a solvent selected from water, ethanol, or a mixture thereof.

13. The pharmaceutical composition according to claim 12, further comprising a cosolvent or excipient.
